(19) United States
(12) Patent Application Publication
(10) Pub. No.: US 2024/0141297 A1
(43) Pub. Date: May 2, 2024

(54) USE OF MESENCHYMAL STEM CELLS OVEREXPRESSING PACER FOR THE TREATMENT OF DISEASES WITH INFLAMMATORY ORIGIN AND/OR COMPONENT

(71) Applicants: Universidad Mayor, Providencia, Santiago (CL); Universidad del Desarrollo, Las Condes, Santiago (CL)

(72) Inventors: Flavio Carrión Arriagada, Las Condes, Santiago (CL); Sebastián Beltrán Vergara, San Miguel, Santiago (CL); Cristian Bergmann Muñoz, Maipú, Santiago (CL); Patricio Manque Manque, Huechuraba, Santiago (CL); Ute Wöehlbier, Huechuraba, Santiago (CL)

(21) Appl. No.: 18/280,093

(22) PCT Filed: Mar. 2, 2022
(86) PCT No.: PCT/CL2022/050020

§ 371 Date: Sep. 1, 2023

Related U.S. Application Data
(60) Provisional application No. 63/155,725, filed on Mar. 2, 2021.

(51) Int. Cl. C12N 5/0775; A61K 35/28; A61P 1/00; A61P 37/06; C07K 14/47; C12N 5/10; C12N 15/86

(52) U.S. Cl. CPC C12N 5/0663; A61K 35/28; A61P 1/00; A61P 37/06; C07K 14/47; C12N 5/10; C12N 15/86; C12N 2740/15043

(57) ABSTRACT

The present invention provides Mesenchymal Stem Cells (MSC) genetically modified for overexpressing Pacer—a newly described autophagy protein—where these MSC have immunomodulatory effects. The invention also provides the method for obtaining the MSC overexpressing Pacer and pharmaceutical formulations comprising said cells.

The MSC according to the invention are useful in the treatment of diseases with inflammatory origin and/or component.

### FIELD OF THE INVENTION

The invention is in the field of cellular therapies and mesenchymal stem cell technologies. Provided herein is a method of treating an individual having or at risk of developing a disease of inflammatory origin and/or component and/or conditions related to the immune system comprised of administering to the individual a therapeutically effective amount of Mesenchymal Stem Cells (MSC) genetically modified for overexpressing Pacer (MSC-Pacer).

### BACKGROUND OF THE INVENTION

Initially, MSCs were used for tissue repair in regenerative medicine; nevertheless, emerging evidence shows that cell therapy using MSC is a powerful therapeutic tool for diseases of inflammatory origin. It is known that the administration of MSC in murine models, as well as in humans, modulates the immune response through the secretion of immunosuppressive factors. However, it also is known that the host's microenvironments modulate the immunosuppressive function of MSC and induces physiological change such as low cellular viability and rapid differentiation of MSC into more specialized cells that do not display immunomodulatory functions. Hence, it is favorable to aim to prolong the survival and stemness of MSC to improve their immunomodulatory and therapeutic effects.

From a therapeutic point of view, immunomodulation refers to any process in which an immune response is altered to the desired level, producing suppression (immunosuppression) or activation of the system (immunoproliferation). MSC have been shown to suppress the immune system, through two principal mechanisms: i) secretion of different growth factors, cytokines and chemokines, and ii) immunosuppression by cell contact. It has been shown that MSC can affect the innate and adaptive immunity system in different ways: including i) suppressing T cell proliferation, cytokine secretion, cytotoxicity and modulating the balance of T helper 1 (Th1)/T helper 2 (Th2) (Duffy et al. 2011); ii) regulating the function of regulatory T cells (Tregs) (Tang et al. 2015); iii) increasing B-cell viability but they also may inhibit B cell proliferation and arrest their cell cycle; in addition, MSC affect the secretion of antibodies and production of co-stimulatory molecules of B cells; iv) inhibiting the maturation, activation and antigen presentation of dendritic cells (DCs); and v) MSCs can also inhibit interleukin-2 (IL-2)-induced Natural Killer (NK) cell activation. Adaptive immunity is a subset of the immune system that is highly specific and protects the host from pathogens or toxins. It is mediated by B and T cells and is characterized by the generation of immunological memory. T cells are widely distributed in both animal and human tissues and once activated, can differentiate into Th1, Th2, Th9, Th17 or Treg subpopulations, according to the intensity of stimulation and the cytokine microenvironment. It has been demonstrated that MSC interact tightly with T cells. Thus, MSC secrete a great diversity of immunosuppressive factors, chemokines, and adhesion molecules, which are responsible for effective T cell suppression, T cell proliferation, apoptosis, and differentiation. For example, MSC are capable of repressing T cell proliferation through cellular or nonspecific mitogenic stimuli and promote apoptosis of activated T cells via the Fas/Fas ligand pathway. In addition, Tregs, as a specialized subset of T cells, restrain the effects of the immune system, leading to relieving their own antigens and sustaining homeostasis. Moreover, MSC contribute to the generation of an immunosuppressive environment via the inhibition of pro-inflammatory T cells and the induction of Tregs cells, as shown in a mouse model for Multiple Sclerosis (MS).

MSC can inhibit B cell proliferation and activation in vitro. MSC also suppress differentiation of B cells, as well as the expression of chemokine receptors owing to cell contact and secretion of soluble molecules. Thus, MSCs suppress antibody production by B cells, and this effect is dependent upon the strength of the inflammatory stimulation, as well as the ratio of MSC to B cells.

Therefore, MSC are excellent candidates for therapeutic use as cellular therapies that can potentially revolutionize the current pharmaceutical landscape.

Several strategies can be used to enhance the immunomodulatory properties of MSC in vitro and in vivo, of which two are widely used i) pro-inflammatory cytokine priming and ii) genetic engineering.

### STATE OF THE ART

MSC have been used in the treatment of experimental animal models of inflammatory and immune disorders. Autologous, allogeneic and even xenogeneic MSC have shown great promise in the treatment of immune-related diseases such as allergic rhinitis, acute and chronic asthma, autoimmune hearing loss, Experimental Autoimmune Encephalomyelitis (EAE), Systemic Lupus Erythematosus (SLE), Graft versus Host Disease (GvHD) and Inflammatory Bowel Disease (IBD).

Macroautophagy (herein referred to as autophagy) is a process of self-degradation and cell survival. The purpose of autophagy is not simply the degradation of materials, but instead, autophagy serves as a dynamic recycling system that produces new energy for cellular renovation, homeostasis, survival and protection in the case of inflammation produced by bacterial infections. However, little is known about the role of autophagy and the immunomodulatory function of MSC.

For example, patent application WO2008100498A2 describes methods of treatment of individuals having an immune-related disease, disorder or condition, for example, inflammatory bowel disease, graft-versus-host disease, multiple sclerosis, rheumatoid arthritis, psoriasis, lupus erythematosus, diabetes, mycosis fungoides (Alibert-Bazin syndrome), or scleroderma using placental stem cells or umbilical cord stem cells, where said placental stem cell is a CD10+, CD34−, CD105+, CD200+ placental stem cell.

On the other hand, publication EP2080140B1 relates in general to the field of diagnostic for monitoring indicators of metastatic melanoma and/or immunosuppression, and more particularly, to a system, method and apparatus for the diagnosis, prognosis and tracking of metastatic melanoma and monitoring indicators of immunosuppression associated with transplant recipients (e.g., liver). There is a brief mention about C13orf18, nevertheless, no connection with the role of Pacer or the use of MSC overexpressing Pacer is addressed in this document.

### Pacer as a New Component of the Autophagy Pathway

Pacer was first described as a Beclin-1 interacting protein and a component of the autophagy pathway by Behrends et al 2010. In 2017, it was reported that Pacer (protein associated with UVRAG as autophagy enhancer), has a role in positively regulating autophagosome maturation (Cheng et al. 2017). Other names for Pacer also have been used in the literature and databases, such as C13orf18, RUBCNL, or KIAA0226L. Recently the group of inventors of the present invention demonstrated for the first time that Pacer is a new regulator of proteostasis and autophagy associated with ALS pathology (Beltran and Nassif et al. 2019). This gene encodes a cysteine-rich protein that contains a putative RING-zinc finger domain (Ring-Zf_9), also referred to Rubicon-homology (RH) domain. Pacer is part of a complex with Beclin1 and UVRAG regulating autophagosome maturation of PI3KC3 and HOPS complex; furthermore, cellular studies showed that Pacer plays a critical role in bacterial infection, hepatic lipid homeostasis, and protein aggregate clearance. In vitro, Pacer deficiency leads to impaired autophagy and accumulation of ALS-associated protein aggregates.

Pacer shares sequence homology with Rubicon, a negative regulator of autophagy, which recently was shown to play a role in immunity and LC3-associated phagocytosis (LAP). Pacer was shown to antagonize Rubicon in autophagy processes.

Although most in vitro studies have highlighted the immunosuppressive properties of MSC, several studies have provided evidence that mismatched MSC are immunogenic. Strategies to prolong MSC persistence and/or to overcome rejection of allo-MSC can be divided into two primary categories: modification of the host and modification of MSC.

Although, studies of the role of autophagy in MSC immunosuppressive function demonstrated that activation of autophagy could improve the immunosuppressive function of MSC (Weiss et al. 2019) and in the state of the art there are a wide variety of methods of treatment with stem cells for inflammatory diseases and/or conditions related to the immune system, up to date, there are no references directed to MSC genetically modified for overexpressing Pacer used for the treatment for diseases with inflammatory component or origin.

### DESCRIPTION OF THE FIGURES

FIG. 1. Determination of overexpression of Pacer in MSC. (A) Western blot of MSC overexpressing Flag-tagged human Pacer (MSC-hPPacer) and MSC control cells carrying an empty vector (MSC-EV). Anti-Flag antibody was used to detect Flag-tagged hPacer. P-Actin was used as a loading reference. (B) mRNA levels of Pacer expressed in MSC-EV and MSC-hPacer were analyzed by qPCR (n=4). For statistical analysis Student t-test was performed. p values: n.s., non-significant, *, p≤0.05; **, p≤0.01; ***, p≤0.001; ****, p≤0.0001.

FIG. 2. Pacer overexpression enhances the immunosuppressive properties of MSC. (A-C) Overexpression of Pacer in MSC transduced with lentiviral particles carrying human Flag-tagged human Pacer (MSC-hPacer) or an empty control vector (MSC-EV). (A) Co-culture assay of CD3-positive (CD3+) splenocytes with MSC-EV or MSC-hPacer compared to wild-type unmodified MSC (n=4). Splenocyte proliferation was induced with 1 μg/ml Concanavalin A (ConA). T-cell proliferation was evaluated by flow cytometry, gating SytoxGreen-negative staining (live CD3+ cells) and assessing Cell Trace Violet (CTV) staining. (B) Percentage of live CD3+ T cells. (C) Percentage of CD3+ T cell proliferation. In (B and C) Mean±SEM are shown. In (B and C) one-way ANOVA with Tukey post-hoc test was performed for statistical analysis. p values: n.s., not significant; *, p≤0.05; **, p≤0.01; ***, p≤0.001; ****, p≤0.0001.

FIG. 3. Determination of ConA concentration for the T cell proliferation assay. (A) Splenocyte, T cell CD3+ and live cell gating. (B) Assessment of ConA concentration dependency (in the range from 0-2 μg ConA) of T cell proliferation using Cell Trace Violet.

FIG. 4. Determination of MSC:splenocyte ratio for the T cell proliferation assay. (A and B) T cells were treated with ConA at a concentration of 0.5 μg and co-cultured with different cell numbers of MSC ranging from 1×104 to 1.5×105. (A) Representative gatings for CD3+ T cell proliferation are shown for each MSC:splenocyte ratio. (B) Percentage of CD3+ T proliferation. In (B) mean±SEM is shown. One-way ANOVA with Tukey post-hoc test was performed for statistical analysis. p values: n.s., not significant; *, p≤0.05; **, p≤0.01; ***, p≤0.001; ****, p≤0.0001.

FIG. 5. hTNFα enhances the immunosuppressive function of MSC. (A) Proliferation assay of splenocytes in co-culture with MSC pretreated or not with hTNFα. Briefly, splenocytes were co-cultured with MSC which were previously treated or not with 10 ng/ml hTNFα for 24 h. Cells were used in a ratio of MSC:splenocytes of 1:10. T cell proliferation was assessed with Cell Trace Violet (CTV) by flow cytometry, gating on CD3+, CD4+, and CD8+ cells. (B) Percentage of viable T-cells. (C) Proliferation percentage of CD3+ T cells. In (B and C) mean±SEM are shown. p values: n.s., non-significant, not significant (n.s). *, p>0.05; **, p<0.05.

FIG. 6. Pacer deficiency in MSCs impairs their immunosuppressive effect on T cells. (A-C) Depletion of endogenous Pacer levels in MSC using siRNA oligos. (A) Co-culture assay of CD3-positive (CD3+) splenocytes with MSC transiently transfected with siPacer or siCtrl oligos (n=7). Wild-type unmodified MSC were used as a reference. Splenocyte proliferation was induced with 1 μg/ml Concanavalin A (ConA). T-cell proliferation was evaluated by flow cytometry, gating SytoxGreen-negative staining (live CD3+ cells) and assessing Cell Trace Violet (CTV) staining. (B) Percentage of CD3+ proliferation cells under ConA (0.5 μg/ml) stimulation. (B) Percentage of live CD3+ T cells. (C) Percentage of CD3+ T cell proliferation. In (B and C) Mean±SEM are shown. In (B and C) one-way ANOVA with Tukey post-hoc test was performed for statistical analysis. p values: n.s., not significant; *, p≤0.05; **, p≤0.01; ***, p≤0.001; ****, p≤0.0001.

FIG. 7. Overexpression of Pacer in MSCs improves their therapeutic effects on DSS-induced inflammatory colon injury. Colitis was induced in C57BL/6 mice by administering 2.5% DSS dissolved in the drinking water from day 0 to 7. Mice receiving MSC-EV and OE-Pacer were injected i.p. at day 3 (with 2×10⁶ MSC per mouse). The control group (Ctrl) corresponded to mice that only received drinking water without DSS. (A) The disease activity index (DAI) scores of Ctrl (n=6), DSS (n=7), DSS+MSC-EV (n=8) and DSS+MSC-hPacer (n=8) was determined daily. The DAI was calculated from cumulative scores for body weight loss, stool consistency and presence of bleeding. (B) Weight loss was assessed daily and plotted as a single parameter, as well as was part of a composite score in (A). (C) The colon length of each experimental group was measured, (D) and results were graphically displayed. In (A) and (B) two-way ANOVA and in (D) one-way ANOVA with Tukey post-hoc test was performed for statistical analysis. Only significant p values are shown. p values: *, p≤0.05; **, p≤0.01; ***, p≤0.001; ****, p≤0.0001.

FIG. 8. Overexpression of Pacer in MSC improves their tissue repair effects during DSS-induced tissue damage. (A) Representative images of colon morphologies from each group (Ctrl, DSS, DSS+MSC-EV and DSS+MSC-hPacer). (B) Histological scoring was performed of images from each animal of each group. For statistical analysis, one-way Anova was performed. p values: n.s., non-significant, *, p≤0.05; ***, p≤0.001; ****, p≤0.0001

### DESCRIPTION OF THE INVENTION

Pacer gain-of function in Mesenchymal Stem Cells enhances its immunosuppressive function by suppressing the proliferation of T cells, and the loss of its function generates the opposite effect, negatively modulating its immunosuppressive capacity.

Interestingly, in cell death trials performed during the development of the invention, it was determined that Pacer overexpression protects these cells from cell death induced by Tumor necrosis factor α (TNFα), and conversely, loss of function decreases cell survival. Therefore, Pacer overexpression in Mesenchymal Stem Cells enhances the immunotherapeutic effect of these by decreasing the signs and clinical symptoms of inflammatory diseases.

Characterization of the genetically modified mesenchymal stem cell overexpressing Pacer.

The present invention provides a genetically modified mesenchymal stem cell wherein said cell is transformed with a vector designed for overexpressing Pacer.

In an embodiment of the invention, the MSC are human mesenchymal stem cells. Although the murine mesenchymal stem cells used during the development of this invention are positive for CD29, CD34, CD44, and Sca-1 (>70%), and negative for CD117 (<5%), the vector can be introduced e.g. in bone marrow derived human MSC (markers e.g. CD73+, CD90+, CD105+, CD166+).

MSC are infected with a lentivirus containing genetic information to express human Pacer, generating MSC transduced with Pacer (MSC-hPacer). Resistant cells are selected.

In the development of the invention in mouse models, the MSC are transduced with Lentiviral particles carrying genetic information for the expression of human Pacer at the 8th to 10th passage. Selection is carried out between 14th to 18th passage. The treatment of mice with modified MSC that overexpress human Pacer is carried out at the 16th to 22th passage.

Types of MSC Used for Overexpressing Pacer.

In an embodiment of the invention, the MSC are derived from bone marrow, adipose tissue, menstrual blood, dental pulp cells, placenta, umbilical cord tissue (Wharton's Jelly) or amniotic fluid, among other sources.

In an embodiment of the invention, the MSC are human MSC isolated from different sources.

MSC-hPacer can be used in the treatment of inflammatory conditions such as cancer, graft-versus-host disease (GvHD), rheumatoid arthritis, psoriasis, systemic lupus erythematosus (SLE), multiple sclerosis (MS), amyotrophic lateral sclerosis (ALS), Sjogren syndrome, systemic sclerosis, inflammatory bowel disease, Crohn's disease or ulcerative colitis (UC).

The present invention also provides a pharmaceutical formulation comprising a suspension of viable MSC genetically modified for overexpressing Pacer (MSC-hPacer) and a pharmaceutically acceptable carrier.

In the development of the invention in mouse models, the cells in the pharmaceutical formulation are in a concentration of 1E+6 to 5E+6 cells per ml.

Optionally, the pharmaceutical formulation comprises pharmaceutically acceptable additives or formulation enhancers.

In a further embodiment of the invention, it is provided a method of treating an individual having or at risk of developing a diseases of inflammatory origin and/or component and/or conditions related to the immune system comprising administering to the individual a therapeutically effective amount of Mesenchymal Stem Cells genetically modified for overexpressing Pacer (MSC-hPacer).

### Results.

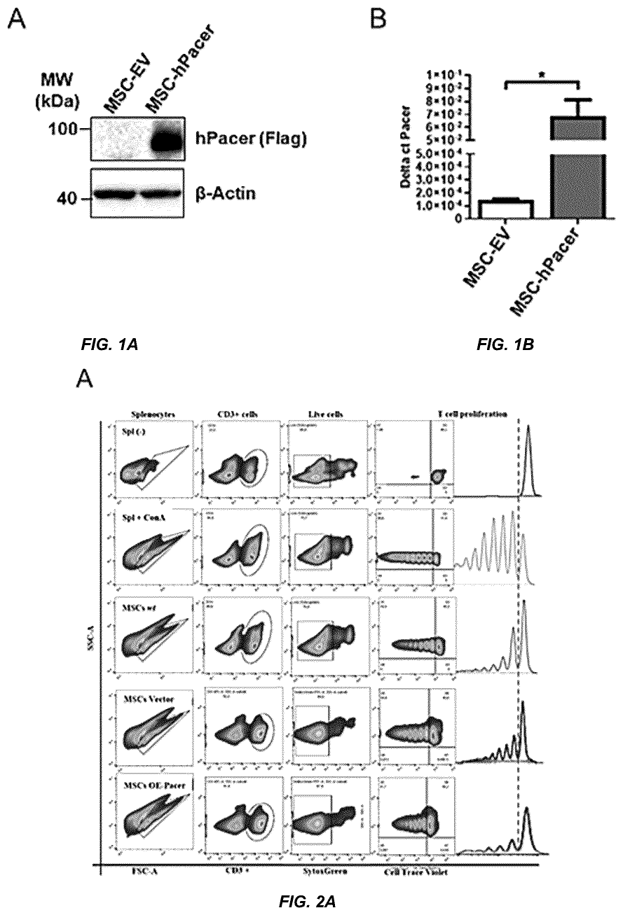

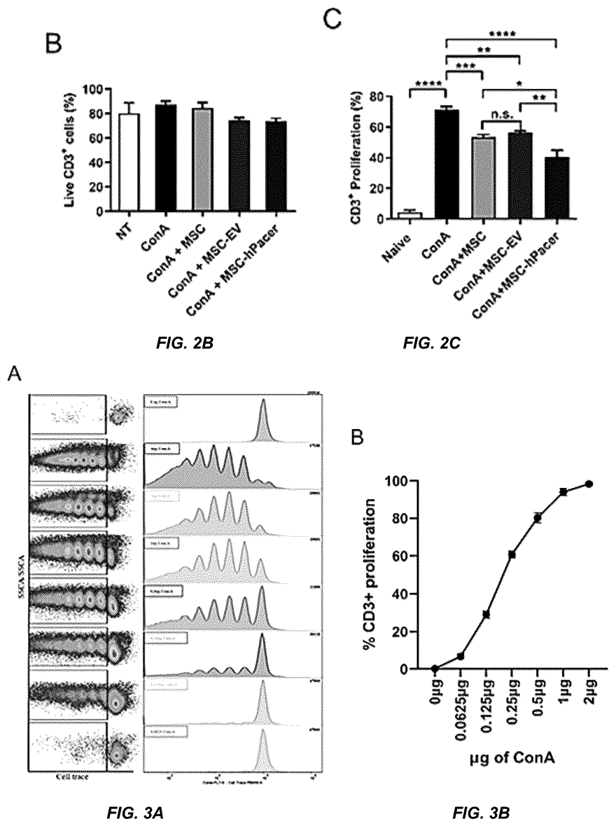

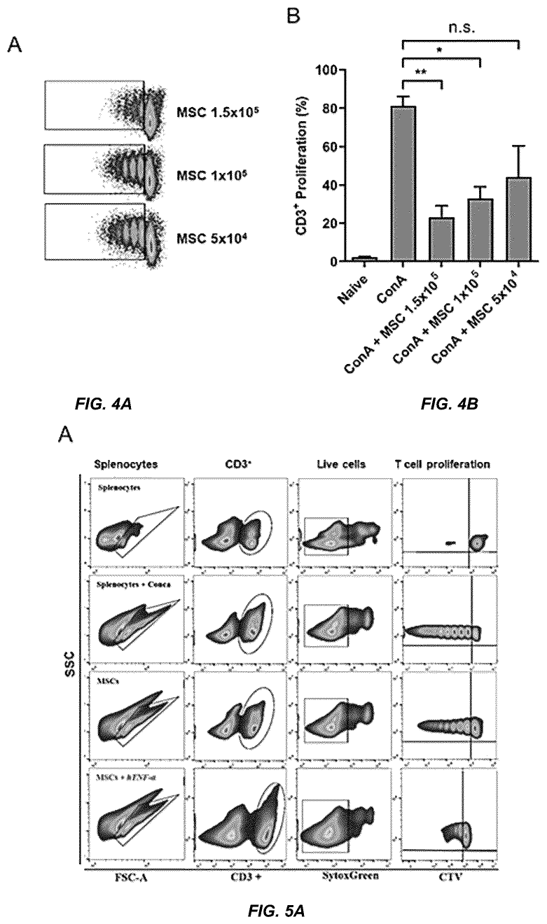

The results showed that the overexpression of human Pacer protein in MSC (FIG. 1) was able to enhance the immunosuppressive functions of MSC (FIG. 2), as well as in their ability to survive (data not shown). The MSC immunosuppressive effect was directly proportional to the concentration of Concanavalin A (ConA) used to stimulate splenocytes (FIG. 3) as well as proportional to the concentration of MSC used in the co-culture (FIG. 4). The optimal MSC/immune cells ratio was within the range of 1:1-1:10 (FIG. 4).

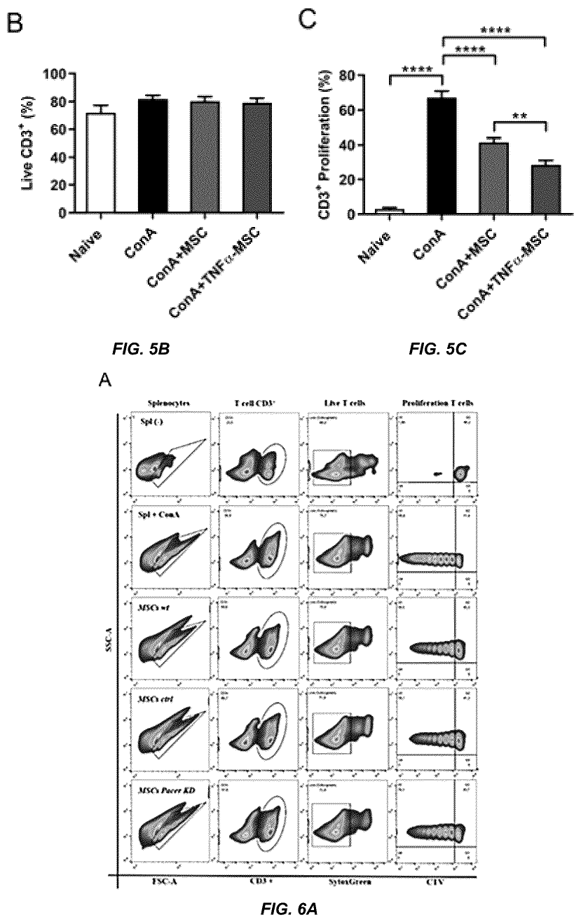

A potent immunosuppressive function of MSC was observed under TNFα stimulation compared with untreated MSC, as suggested by significantly reduced T cell proliferation (FIG. 5).

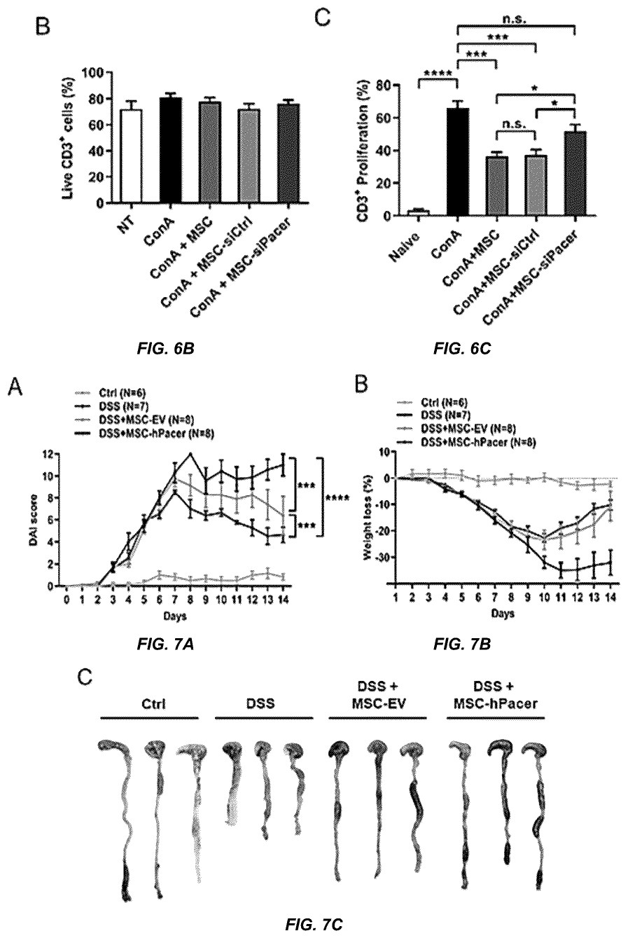

Furthermore, it was found that the depletion of Pacer in MSC impairs their immunosuppressive function over T cell proliferation (FIG. 6), while overexpression of Pacer in MSC enhances their immunosuppressive function, decreasing CD3+ T cell proliferation. Together these results suggest that Pacer may be involved in the regulation of the immunomodulatory capacity of MSCs.

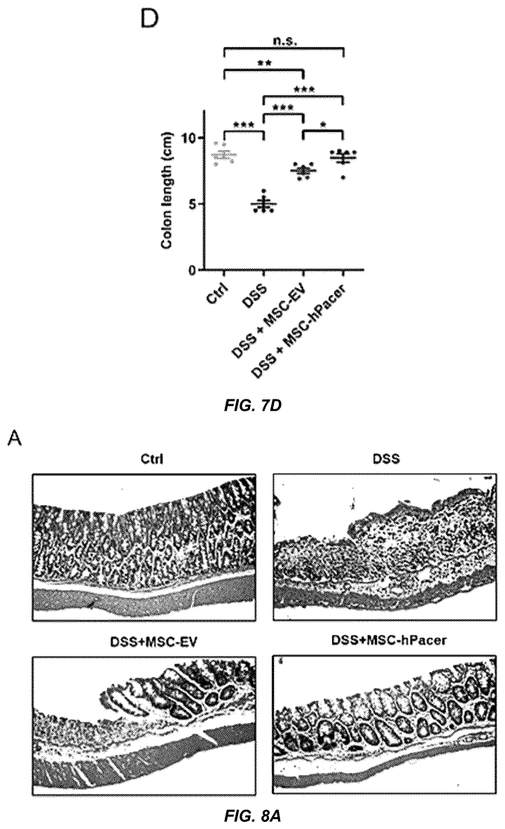

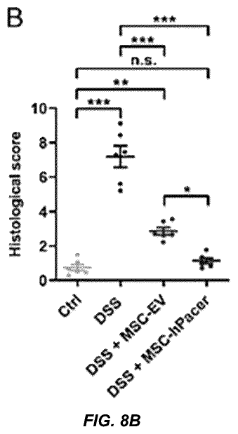

Finally, increased expression of Pacer in MSC enhanced their ability to ameliorate the symptoms of DSS-induced colitis in mice (FIGS. 7 and 8). In agreement with the observations in vitro, the results showed that Pacer overexpression in MSC improved the therapeutic effect of MSC in a DSS-induced inflammatory colon injury model (FIG. 7). Furthermore, this therapeutic effect was accompanied not only by ameliorating the clinical score (FIG. 7A), but also an improved recovery of the intestinal epithelium and therefore also its functionality (FIG. 8).

### EXAMPLES

### Example 1: Genetic Modification of MSC to Overexpress Pacer

### Plasmid Constructs and Lentivirus Vector Construction

pLenti-C-Myc-DDK-P2A; pLenti-C-C13orf18-Myc-DDK-P2A; mPacer shRNA; shPacer B constructs were synthesized by and purchased from Origene. All the plasmids were prepared with the Qiagen plasmid midi kit (Qiagen,) according to the manufacturer's instructions. Lentiviral particles were produced in HEK 293T cells. Briefly, HEK293T cells were seeded at a concentration 2.5E+6 in a 10 cm dish in 10 ml complete DMEM growth media (without antibiotic) and incubated overnight. Then the cells were transfected with 5 μg of either empty vector or human Pacer Flag-tagged plus 6 μg of packaging plasmids from Lenti-ORF clones kit (Origene, TR30022). The medium was replaced 12 h post transfection. The viral supernatant was collected at 24 h and 48 h and filtered through a 0.45 μm filter to remove cellular debris.

High titer lentiviral stocks were produced (1E+6 to 1E+7 TU/ml), and MSC at the 9th passage were infected with empty vector control lentivirus (MSC-EV) or lentivirus expressing Pacer (MSC-hPacer) according to the manufacturer's protocol. Cells resistant to puromycin (10 μg/ml) were selected at the 16th passage and used at the 18-20th passages.

### Example 2: Pacer Overexpression Enhances Immunosuppressive Properties of MSCs

To determine the role of the Pacer in MSC-mediated immunomodulatory effects, gain of function experiments were performed. Thus, MSC overexpressing human Pacer (MSC-hPacer) and empty vector control cells (MSC-EV) were generated by lentiviral transduction. The overexpression of Pacer in MSC was confirmed by Western blot and real time PCR (FIGS. 1A and 1B, respectively).

Due to the results with Pacer depletion, which suggested a role for Pacer in mediating MSC inhibitory effects on T-cell proliferation, it was investigated whether MSC-hPacer display improved immunosuppressive function in a splenocyte co-culture assay. The results show that MSC-hPacer suppress the proliferation of activated T-cells more efficiently than MSC-EV or wild-type unmodified MSC (FIGS. 2A and C). Hence, Pacer overexpression leads to an increase in the immunosuppressive properties of MSC evident by a decrease of CD3+ T-cell proliferation when compared to empty vector controls cells (FIG. 2C). No changes in the viability of T cells were observed (FIG. 2 b). Together these results advocate for an immunosuppressive effect of Pacer in MSC towards T cell proliferation suggesting a potential therapeutic application of modulating Pacer levels in MSC for inflammatory disease treatment.

### Example 3: In Vitro T Cell Proliferation Assay

To study the immunosuppressive potential of genetically modified MSC, freshly isolated splenocytes (1×10⁶ cells/well) derived from C57BL/6 mice were labeled with 10 μM CellTrace™ Violet (CTV) (Invitrogen, UK) according to the manufacturer's instructions and were stimulated with Concanavalin A (ConA) (1 μg/ml) and co-cultured with MSC at a ratio of 10:1 (splenocyte:MSCs) for five days, then collected for flow cytometric analysis using a CytoFLEX (Beckman Coulter, USA).

In a dose-response experiment using different concentrations of ConA, it was defined that 1 μg/ml of ConA is the optimal concentration, which resulted in around 90% of T cell proliferation (FIG. 3).

Next, it the optimal ratio MSC:Splenocyte was determined for the assay. Therefore, we used 3 different ratios MSC:splenocytes (1.5:10; 1:10; 0.5:10) and showed that the optimal concentration MSC:splenocytes is 1:10, which decreased the CD3+ T cell proliferation by about 40% (FIG. 4), concluding that the optimal ratio for the next experiments is 1:10 MSC:Splenocytes with 1 μg/ml of ConA stimulation.

Next, the proliferation percentage of CD3+ cells stimulated with ConA in the presence or not of MSC treated with TNFα was determined. The results showed that MSC stimulated with hTNFα suppressed the proliferation of T cells more efficiently when compared with those that were not stimulated (FIGS. 5A and 5C). Furthermore, the percentage of live CD3+ T cells was measured, however, no significant differences between the groups was found (FIG. 5B).

### Example 4: Pacer Deficiency in MSCs Impairs their Immunosuppressive Effect on T Cells

Knockdown of Pacer through siRNA technology was performed. MSC were seeded at 2×10⁵ cell/well in six-well plates and transfected 24 h later with ON-TARGET plus smart-pool siRNAs (Dharmacon,) using Dharmafect Transfection Reagents (Dharmacon). Dharmafect was used at 4 μl for a final concentration of 30 nM siRNA/well. Non-targeting siRNA (NT) was used as a control siRNA.

To determine the role of Pacer in MSC-mediated immunomodulatory effects, “loss of function” experiments by depleting Pacer in MSC using RNA interference (siRNA) were performed. The successful knockdown of Pacer in MSC (here called MSC-siPacer) compared to siRNA oligos control (here called MSC-siCtrl) was evaluated by Western blot and real time qPCR (data not shown). Furthermore, for all the experiment wild-type MSC were used as a reference (referred to as MSC).

The results showed that the co-culture of MSC-siPacer with CD3+ T cells led to a significantly lower inhibitory effect in their proliferative ability (FIGS. 6A and 6C). Indeed, the percentage of T cell proliferation was increased in cells incubated with MSC-siPacer (FIG. 6C). As expected, substantial suppression was observed in the co-cultures of T cells with wild-type MSC or MSCsiCtrl, with no differences observed between both groups (FIGS. 6A and 6C). No changes in the survival of cells were observed suggesting that the effects are most likely due to the changes in the expression of Pacer. This result demonstrated that the loss of endogenous Pacer in MSC impairs their immunosuppressive function.

### Example 5: DSS-Induced Inflammatory Bowel Injury in Mice

As described above Pacer upregulation enhanced the immunosuppressive abilities of MSC in vitro, thus an acute inflammatory colitis injury mouse model induced by the administration of dextran sulfate sodium (DSS) was used to demonstrate the therapeutic effect of MSC-hPacer in vivo.

Briefly, 2.5% Dextran Sulfate Sodium (DSS) dissolved in drinking water was orally administered to female C57BL/6 mice (14-16 weeks old) to induce inflammatory bowel injury for 14 days. MSC (1×10⁶) were intraperitoneally (i.p.) administrated to mice 3 days after oral administration of DSS. Mice were observed daily for weight loss, stool consistency/diarrhea, and the presence of rectal bleeding. The Disease Activity Index (DAI) was calculated using the following parameters described previously (Naito et al. 2004): i) weight loss (0-4); (ii) stool consistency/diarrhea (0-4) and (iii) rectal bleeding (0-4). We calculated a total score of DAI: 0 (healthy mice) to 12 (severe colitis). Mice were sacrificed at day 14, and colon length was measured. Subsequently, serum and intestinal tissues were sampled. 4% paraformaldehyde-fixed intestine histological sections were stained with hematoxylin and eosin.

Mice received DSS orally, causing the death of epithelial cells in the colon, compromising the barrier function and causing subsequent inflammation. To explore the effect of MSC-hPacer in vivo, MSC-EV and MSC-hPacer were i.p. injected into the mice on day 3 after DSS administration. The intestinal injury was significantly ameliorated in mice that received MSC-hPacer compared with mice that received MSC-EV (FIG. 7A). Furthermore, mice treated with MSC-EV and MSC-hPacer both recovered weight in the last 3 days of the experiment, however the MSC-hPacer treated animals recovered their weight to a greater although not significant extent than the MSC-EV treated animals (FIG. 7B). Large intestine and cecum were extracted from the peritoneal cavity of mice. To determine if MSC-hPacer treatment affected overall colon physiology/morphology we measured the colon length (FIGS. 8C and 8D). MSC-hPacer treated mice had a similar colon length compared to healthy control mice and improved colon length compared to MSC-EV treated mice. These results suggest that MSC-hPacer show improved capabilities to alleviate DSS-induced inflammatory colon injury compared to non-modified MSC.

Histological analysis of hematoxylin/eosin-stained colon sections showed that in animals treated with MSC-hPacer the intestinal epithelium recovered to a significantly greater extent compared to animals treated with MSC-EV (FIGS. 8A and 8B). Furthermore, lower levels of inflammation with scattered infiltrating mononuclear cells (1-2 foci) was observed in MSC-hPacer treated mice compared to MSC-EV treated mice (FIG. 8A).

1. A genetically modified mesenchymal stem cell wherein said cell is transformed with a vector containing genetic information to express human Pacer.

2. The genetically modified mesenchymal stem cell according to claim 1, where the MSC is obtained from bone marrow, adipose tissue, menstrual blood cells, dental pulp cells, placenta, umbilical cord tissue or amniotic fluid.

3. The genetically modified mesenchymal stem cell according to claim 1, where the MSC is human mesenchymal stem cell.

4. The genetically modified mesenchymal stem cell according to claim 3, where the MSC is bone marrow derived human MSC CD73+, CD90+, CD105+ and CD166+.

5. A pharmaceutical formulation comprising a suspension of viable Mesenchymal Stem Cells genetically modified for overexpressing Pacer (MSC-hPacer) and a pharmaceutically acceptable carrier.

6. A pharmaceutical formulation according to claim 5 additionally comprising pharmaceutically acceptable additives or formulation enhancers.

7. Use of Mesenchymal Stem Cells genetically modified for overexpressing Pacer (MSC-hPacer) for the treatment of diseases of inflammatory component and/or origin and/or conditions related to the immune system.

8. Use according to claim 7 where the inflammatory condition is a viral or bacterial infection, cancer, graft-versus-host disease (GvHD), rheumatoid arthritis, psoriasis, systemic lupus erythematosus (SLE), multiple sclerosis (MS), amyotrophic lateral sclerosis (ALS), Sjogren syndrome, systemic sclerosis, inflammatory bowel disease, Crohn's disease or ulcerative colitis (UC).

9. A method of treating an individual having or at risk of developing a disease of inflammatory origin and/or component and/or conditions related to the immune system comprising administering to the individual a therapeutically effective amount of Mesenchymal Stem Cells genetically modified for overexpressing Pacer (MSC-hPacer).

10. The method according to claim 9 wherein said MSC-hPacer is obtained from human bone marrow, adipose tissue, menstrual blood cells, dental pulp cells, placenta, umbilical cord tissue or amniotic fluid.
